(19) United States
(12) Patent Application Publication
(10) Pub. No.: US 2025/0114078 A1
(43) Pub. Date: Apr. 10, 2025

(54) ULTRASOUND DIAGNOSTIC APPARATUS, ULTRASOUND DIAGNOSTIC SYSTEM, POWER SOURCE CONTROL METHOD, AND NON-TRANSITORY COMPUTER READABLE STORAGE MEDIUM

(71) Applicant: Konica Minolta, Inc., Tokyo (JP)

(72) Inventor: Morio NISHIGAKI, Kanagawa (JP)

(21) Appl. No.: 18/899,438

(22) Filed: Sep. 27, 2024

(30) Foreign Application Priority Data
Oct. 5, 2023 (JP) 2023-173651

(51) Int. Cl. A61B 8/00

(52) U.S. Cl. CPC A61B 8/56; A61B 8/4405; A61B 8/54

(57) ABSTRACT

An ultrasound diagnostic apparatus includes: an executor that executes ultrasound diagnosis; a battery that supplies electric power to the executor, and a hardware processor that is connectable to both a first power source, which supplies the electric power to the executor and the battery, and a second power source, which supplies the electric power to the executor and the battery and has suppliable electric power larger than suppliable electric power of the first power source, and performs control such that amounts or an amount of the electric power to be supplied to the executor and/or the battery are/is varied according to electric power supplying capability of a power source that is the first power source or the second power source and has been connected.

### CROSS REFERENCE TO RELATED APPLICATIONS

The entire disclosure of Japanese Patent Application No. 2023-173651, filed on Oct. 5, 2023, is incorporated herein by reference in its entirety.

### BACKGROUND

### Technological Field

The present invention relates to an ultrasound diagnostic apparatus. an ultrasound diagnostic system, a power source control method, and a non-transitory computer-readable storage medium.

### Description of Related Art

In recent years, there has been a portable ultrasound diagnostic apparatus that is easy for a user to carry and use to a place where ultrasound diagnosis is performed. Such an ultrasound diagnostic apparatus can be operated by supply of electric power from an external power source or supply of electric power from an internal battery charged by supply of electric power from an external power source.

Since the capacity of a battery is limited in a portable ultrasound diagnostic apparatus, the time available for diagnosis is shorter than in a stationary ultrasound diagnostic apparatus which is larger and in which a large-capacity battery is generally mounted. When the remaining capacity of the battery becomes insufficient during the diagnosis, the diagnosis is interrupted, and in addition, the setting and the like used for the diagnosis are reset. Even when the ultrasound diagnostic apparatus is connected to another power source and restarted, resetting, redoing of diagnosis, or the like occurs in order to restart diagnosis, and thus a large amount of labor may be required.

Regarding power source control in an ultrasound diagnostic apparatus, there are known technologies described in, for example, Japanese Patent Publication Laid-Open No. 2018-166967 and Japanese Patent Publication Laid-Open No. 2016-146890. Japanese Patent Publication Laid-Open No. 2018-166967 describes a medical image diagnostic apparatus which includes a power source capable of supplying electric power with a large electric current by improving efficacy with a power-factor improvement circuit. Japanese Patent Publication Laid-Open No. 2016-146890 describes an ultrasound diagnostic system including two power source supplying ports.

### SUMMARY

However, in the technology described in Japanese Patent Publication Laid-Open No. 2018-166967, the amount of electric power to be supplied is constant. That is, in the technology described in Japanese Patent Publication Laid-Open No. 2018-166967, when the battery is charged, a large electric current always flows even when it is not necessarily required. Furthermore, in the technology described in Japanese Patent Publication Laid-Open No. 2016-146890, although power source supplying ports are provided in two positions, it is not possible to supply electric power from the two positions at the same time, and the amount of electric power to be supplied cannot be increased to a large electric current. That is, the technology described in Japanese Patent Publication Laid-Open No. 2016-146890 is not capable of increasing the amount of electric power to be supplied, when it is necessary to charge the battery in a short time or the like. Therefore, both of the technologies described in Japanese Patent Publication Laid-Open No. 2018-166967 and Japanese Patent Publication Laid-Open No. 2016-146890 have a problem in terms of enabling flexible battery charging according to a use situation of a user.

For example, some users can tolerate a long charging time when not in use because, for example, they have a spare ultrasound diagnostic apparatus or use it for a short time, while other users want to complete charging in a short time.

Furthermore, for example, some users tend to use battery-driven apparatuses for a long time and require a large-capacity battery. Use of a large-capacity battery inevitably increases the time required for full charge. Some conventional ultrasound diagnostic apparatuses can allocate part of supply of electric power from an external power source to battery charging while operating an execution section, which executes ultrasound diagnosis, by the supply of electric power from the external power source. In that case, most of the supply of electric power from the external power source is always allocated to the supply of electric power to the execution section, so that the time required for battery charging becomes long. For a user of such an apparatus, it is desirable to shorten a charging time in a case where battery charging is performed while ultrasound diagnosis is performed.

A similar problem may occur depending on the operation mode used by a user. For example, in an operation mode in which a synthetic aperture beamformer is used or in a shear wave generation mode, power consumption increases, and as a result, power that can be allocated to battery charging performed at the same time as the operation of the apparatus decreases. Therefore, the time required for charging the battery becomes longer. Also for such a user, it is desirable to reduce the charging time in a case where battery charging is performed while ultrasound diagnosis is performed.

However, needs such as a reduction in charging time and an increase in capacity of a battery are not common to all users, and are different depending on use situations of users.

An object of the present invention is to provide an ultrasound diagnostic apparatus, an ultrasound diagnostic system, a power source control method, and a non-transitory computer-readable storage medium that enable flexible battery charging according to a use situation of a user.

In order to realize at least one of the above-described objects, an ultrasound diagnostic apparatus reflecting one aspect of the present invention includes: an executor that executes ultrasound diagnosis; a battery that supplies electric power to the executor; and a hardware processor that is connectable to both a first power source, which supplies the electric power to the executor and the battery, and a second power source, which supplies the electric power to the executor and the battery and has suppliable electric power larger than suppliable electric power of the first power source, and performs control such that amounts or an amount of the electric power to be supplied to the executor and/or the battery are/is varied according to electric power supplying capability of a power source that is the first power source or the second power source and has been connected.

An ultrasound diagnostic system reflecting another aspect of the present invention includes: the ultrasound diagnostic apparatus described above; and a cart attachable to and detachable from a main body of the ultrasound diagnostic apparatus. The main body includes the battery as a first battery, the cart includes a second battery, and the hardware processor performs control such that amounts or an amount of the electric power to be supplied to the executor, the first battery, and/or the second battery are/is varied according to the electric power supplying capability of the power source that is the first power source or the second power source and has been connected.

An aspect of a power source control method reflecting yet another aspect of the present invention is a power source control method for an ultrasound diagnostic apparatus including: an executor that executes ultrasound diagnosis; a battery that supplies electric power to the executor; and a main body that is connectable to both a first power source. which supplies the electric power to the executor and the battery, and a second power source, which supplies the electric power to the executor and the battery and has suppliable electric power larger than suppliable electric power of the first power source. The power source control method includes performing control such that amounts or an amount of the electric power to be supplied to the executor and/or the battery are/is varied according to electric power supplying capability of a power source that is the first power source or the second power source and has been connected.

A non-transitory computer-readable storage medium reflecting still another aspect of the present invention stores a program for causing the ultrasound diagnostic apparatus described above to execute the power source control method described above.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is an external perspective view illustrating a front of an ultrasound diagnostic system including an ultrasound diagnostic apparatus according to Embodiment 1;

FIG. 2 is an external perspective view illustrating a rear of the ultrasound diagnostic system including the ultrasound diagnostic apparatus according to Embodiment 1;

FIG. 3 is a block diagram schematically illustrating an internal configuration of the ultrasound diagnostic system including the ultrasound diagnostic apparatus according to Embodiment 1;

FIG. 4 is a block diagram illustrating the internal configuration of the ultrasound diagnostic system including the ultrasound diagnostic apparatus according to Embodiment 1;

FIG. 5 is a block diagram illustrating an internal configuration of an ultrasound diagnostic system including an ultrasound diagnostic apparatus according to Embodiment 2;

FIG. 6 is a block diagram illustrating an internal configuration of an ultrasound diagnostic system including an ultrasound diagnostic apparatus according to Embodiment 3; and

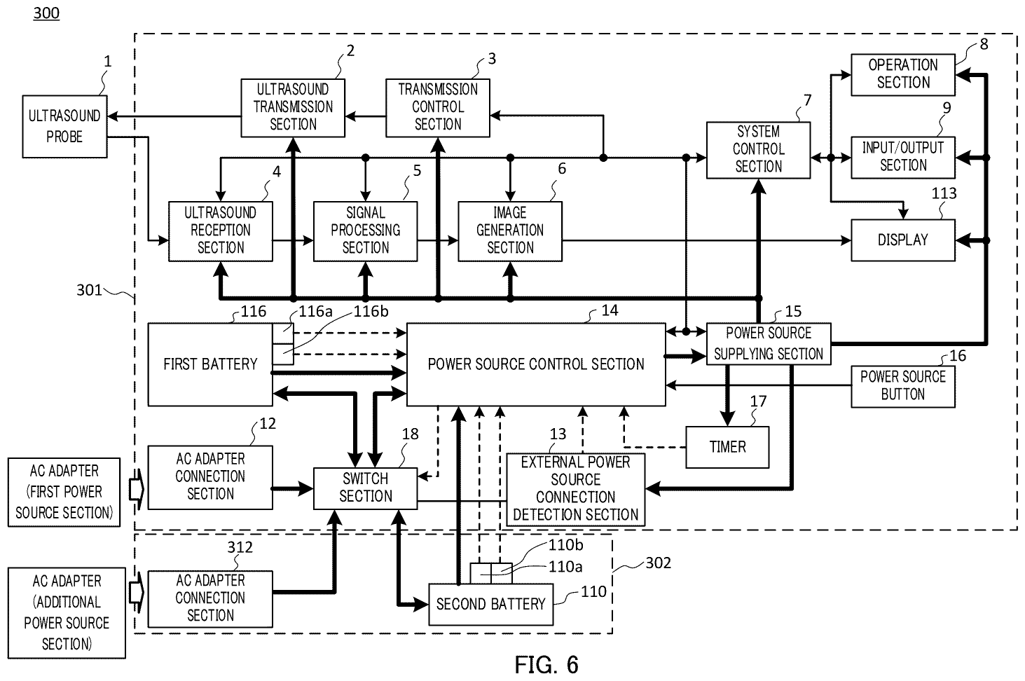

FIG. 7 is a block diagram illustrating an internal configuration of an ultrasound diagnostic system including an ultrasound diagnostic apparatus according to Embodiment 4.

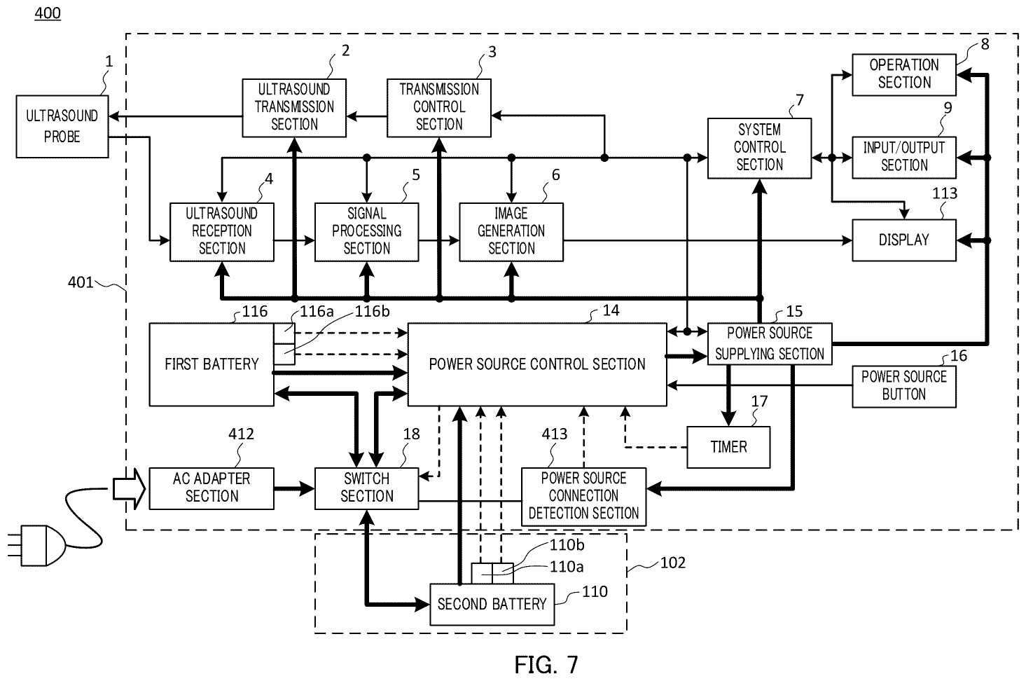

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, embodiments of the present invention will be described in detail with reference to the drawings.

### Embodiment 1

### Appearance Configuration of Ultrasound Diagnostic Apparatus and Ultrasound Diagnostic System

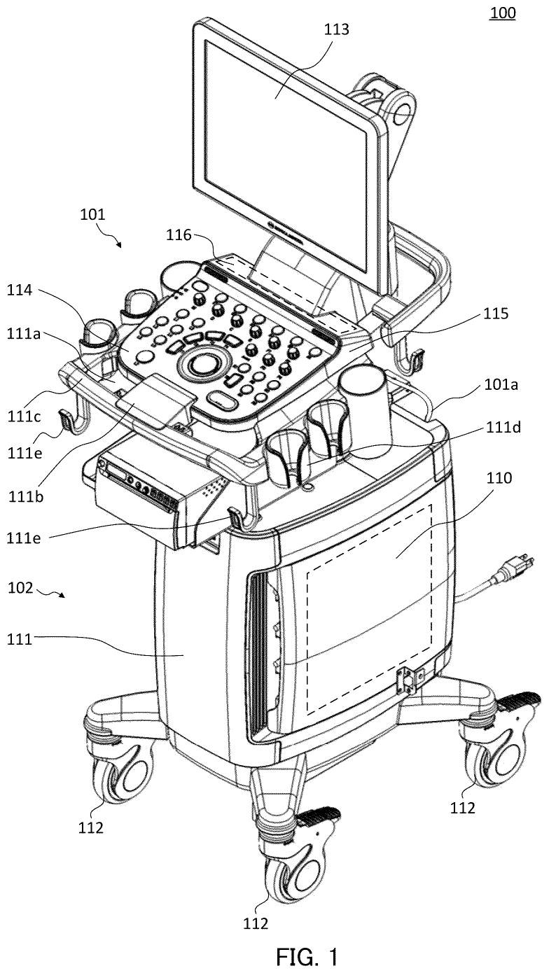

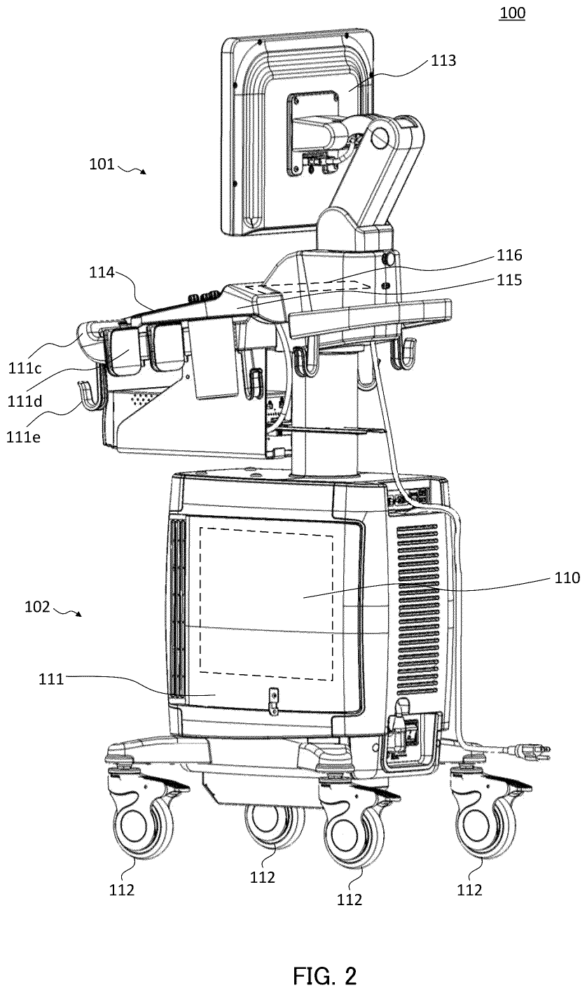

FIGS. 1 and 2 are external perspective views illustrating a front and a rear, respectively, of an ultrasound diagnostic system including an ultrasound diagnostic apparatus according to Embodiment 1 of the present invention.

Ultrasound diagnostic system 100 includes ultrasound diagnostic apparatus 101 and cart 102 on which ultrasound diagnostic apparatus 101 can be placed and to and from which ultrasound diagnostic apparatus 101 is attachable and detachable. Ultrasound diagnostic apparatus 101 is an example of a portable ultrasound diagnostic apparatus that can be conveyed to and used at a place where a user wants to perform ultrasound diagnosis.

Cart 102 includes a housing 111 and caster sections 112 disposed at a lower surface part of housing 111. Attachable/detachable base 111a in which ultrasound diagnostic apparatus 101 is attachably/detachably mounted is provided above housing 111. Second battery 110 is provided outside ultrasound diagnostic apparatus 101. but functions as a power source for ultrasound diagnostic apparatus 101. In the present embodiment, second battery 110 that is incorporated in housing 111 may be configured to be replaceable with a spare battery. Note that, ultrasound diagnostic apparatus 101 of the present embodiment can be operated by supply electric power from an external power source or supply of electric power from first battery 116 (to be described later) even when housing 111 does not incorporate second battery 110.

Ultrasound diagnostic apparatus 101 includes display 113, operation panel 114, apparatus main body 115 (an example of a main body), and first battery 116. Display 113 is connected to attachable/detachable base 111a via an arm and is disposed above attachable/detachable base 111a. Operation panel 114 is mounted in an upper part of attachable/detachable base 111a. Apparatus main body 115 includes operation panel 114 on the main surface. First battery 116 is incorporated in apparatus main body 115. Apparatus main body 115 is held by fixing tool 111b on the front surface side, in a state of being engaged with a root portion of the arm on the rear surface side, and is fixed to attachable/detachable base 111a. First battery 116 is provided inside ultrasound diagnostic apparatus 101 and functions as a power source for ultrasound diagnostic apparatus 101. First battery 116 may be configured to be replaceable with a spare battery.

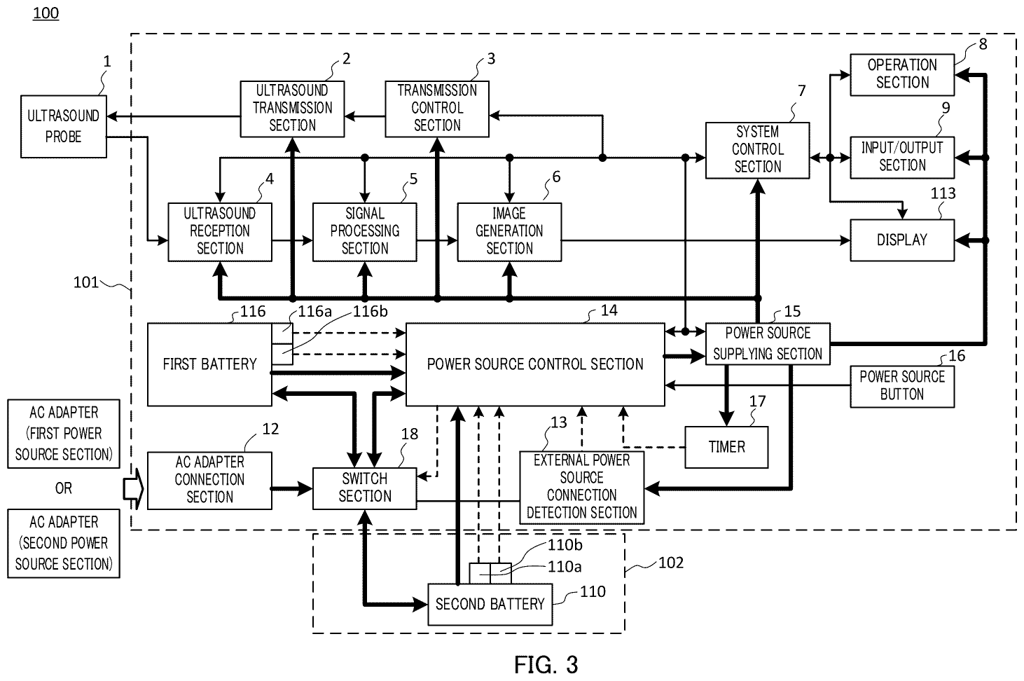

Housing 111 is a storage box for storing second battery 110. Housing 111 has, for example, a substantially rectangular parallelepiped shape. Housing 111 is provided with handle section 111c serving as a handle when ultrasound diagnostic apparatus 101 is operated by being rotated or is operated by being attached/detached. Housing 111 is provided with probe holders 111d for mounting ultrasound probe 1 (see FIG. 3). Housing 111 is provided with a plurality of hook sections 111e with which a cable (not illustrated) for connecting ultrasound probe 1 to apparatus main body 115 is hooked.

Caster sections 112 are provided at the lower surface part of housing 111 and enable ultrasound diagnostic system 100 to move with respect to the floor surface. In the lower surface part of housing 111, for example, caster sections 112 are disposed at the four corners, respectively. Caster section 112 includes, for example, a caster lock constituting a drum brake, and is configured to be changeable between a locked state and an unlocked state by an operation of a user. Caster section 112 is movable (conveyable) in the unlocked state and is fixed in position in the locked state.

Display 113 displays an ultrasound image, a setting screen, and various kinds of information. Operation panel 114 receives an input operation from a user. Apparatus main body 115 in which display 113 and operation panel 114 are integrated is supported by attachable/detachable base 111a protruding upward from housing 111. Since attachable/detachable base 111a is turnable around an axis as the up-down direction, apparatus main body 115 in which display 113 and operation panel 114 are integrated is turnable in the horizontal direction with the turn of attachable/detachable base 111a. Note that, housing 111 is provided with a turning mechanism (e.g., a bearing mechanism) that turnably supports attachable/detachable base 111a with respect to housing 111.

That is, ultrasound diagnostic apparatus 101 is turnable around the axis with respect to housing 111 by a manual operation of a user. A user can freely change the orientation of ultrasound diagnostic apparatus 101 according to the use environment. Note that, the turning mechanism includes a lock member (not illustrated) that locks the pivoting of attachable/detachable base 111a, and is normally held in a state where the pivoting of attachable/detachable base 111a is locked. Furthermore, when the lock of the lock member is released by an operation of a user, ultrasound diagnostic apparatus 101 becomes turnable.

Apparatus main body 115 is attachable to and detachable from housing 111 (attachable/detachable base 111a) by a manual operation of a user. That is, a user can detach apparatus main body 115 from cart 102 according to the use environment. Note that, in a case where apparatus main body 115 is placed on attachable/detachable base 111a for use, apparatus main body 115 is held in a state in which fixing tool 111b of attachable/detachable base 111a is locked. When the lock of fixing tool 111b is released by an operation of a user, apparatus main body 115 can be separated from cart 102.

Ultrasound probe 1 includes a case housing an element array arranged in a two-dimensional array in the longitudinal and transverse directions at a leading-end part of ultrasound probe 1.

Ultrasound probe 1 is configured to include a probe main body, a connection terminal, and a cable. The probe main body is constituted by covering an element array and the like with a resin cover. The connection terminal is connected to a probe connector provided in ultrasound diagnostic apparatus 101 (apparatus main body 115). The cable extends between the probe main body and the connection terminal to connect ultrasound probe 1 to apparatus main body 115.

At the time of ultrasound inspection, ultrasound probe 1 is used in a state in which the connection terminal is connected to the probe connector and the probe main body is held by a user.

Note that, there are various types of probes such as a convex probe and a linear probe as the type of ultrasound probe 1, and in general, these are used differently according to the object or purpose of inspection.

Ultrasound diagnostic apparatus 101 includes first battery 116 that is a battery incorporated in apparatus main body 115. Furthermore, a power source plug to be connected to a commercial power source is connected to ultrasound diagnostic apparatus 101. The power source plug is provided at a terminal end part of a power source cable including an AC adapter (a first power source section or a second power source section) in the middle. When the power source cable is in use, its end part without the power source plug is connected to a power connector in an exposed portion of a lower surface part of ultrasound diagnostic apparatus 101 attached to cart 102, and the power source plug is connected to the commercial power source.

Cart 102 includes second battery 110 which is an external battery attachable to and detachable from ultrasound diagnostic apparatus 101 and has a capacity larger than that of first battery 116 of ultrasound diagnostic apparatus 101. In the present embodiment, no AC adapter is connected to cart 102.

Each of first battery 116 and second battery 110 is replaceable with a spare battery. Ultrasound diagnostic apparatus 101 is driven by at least one of electric power to be supplied from an external power source and electric power discharged from first battery 116 and second battery 110.

### Ultrasound Diagnostic Apparatus and Internal Configuration of Ultrasound Diagnostic System

FIG. 3 is a block diagram schematically illustrating an internal configuration of an ultrasound diagnostic system including the ultrasound diagnostic apparatus according to the present embodiment.

Ultrasound diagnostic apparatus 101 includes ultrasound transmission section 2, transmission control section 3, ultrasound reception section 4, signal processing section 5, image generation section 6, system control section 7, operation section 8, and input/output section 9. Ultrasound diagnostic apparatus 101 further includes alternate current (AC) adapter connection section 12, external power source connection detection section 13, power source control section 14, power source supplying section 15, power source button 16, timer 17, switch section 18, display 113, and first battery 116. Among these constituent elements, ultrasound transmission section 2, transmission control section 3, ultrasound reception section 4, signal processing section 5, image generation section 6, system control section 7, input/output section 9, AC adapter connection section 12, external power source connection detection section 13, power source control section 14, power source supplying section 15, power source button 16, timer 17, switch section 18, and first battery 116 are incorporated in apparatus main body 115 that is portable. Ultrasound diagnostic apparatus 101 is configured to be connectable to ultrasound probe 1 and the AC adapter (the first power source section or the second power source section). Cart 102 includes second battery 110. In ultrasound diagnostic apparatus 101, a combination of ultrasound transmission section 2, transmission control section 3, ultrasound reception section 4, signal processing section 5, image generation section 6, system control section 7, operation section 8, input/output section 9, and display 113 is a main block of ultrasound diagnostic apparatus 101, that is, an example of an execution section that executes ultrasound diagnosis.

Power source control section 14 includes an arithmetic section and a storage section. The arithmetic section is constituted by, for example, a micro processor unit (MPU). The MPU is an example of a hardware processor included in ultrasound diagnostic system 100 for controlling the operation of each section of ultrasound diagnostic system 100. The storage section is, for example, a memory constituted by a storage apparatus such as a non-volatile memory or a hard disk. The memory is an example of a non-transitory computer-readable storage medium that stores a program for realizing, by causing the MPU to execute, the operation of each section of ultrasound diagnostic system 100, specifically, a power source control method described in the present embodiment.

Moreover, power source control section 14 further includes a wiring section for transmitting electric power supplied from first battery 116, second battery 110 or the external power source via switch section 18 to power source supplying section 15. In addition, power source control section 14 further includes a wiring section for transmitting electric power discharged from first battery 116 to second battery 110 via switch section 18 in a case where second battery 110 is charged with the electric power discharged from first battery 116. Power source control section 14 further includes a wiring section for transmitting a current discharged from second battery 110 to first battery 116 via switch section 18 in a case where first battery 116 is charged with the electric power discharged from second battery 110.

Switch section 18 switches the supply of driving electric power to ultrasound diagnostic apparatus 101 and also switches charging to first battery 116 or second battery 110. Switch section 18 and power source control section 14 constitute a main part (an example of a control section) of a charge/discharge switching mechanism in ultrasound diagnostic system 100. The charge/discharge switching mechanism will be described later.

Power source supplying section 15 is an electric circuit including a DC-DC converter or the like, which receives electric power transmitted from power source control section 14, converts the received electric power into electric power suitable for each constituent section in ultrasound diagnostic apparatus 101 based on the control of power source control section 14, and supplies the electric power to each constituent section. The constituent sections in ultrasound diagnostic apparatus 101 include, for example, ultrasound transmission section 2, transmission control section 3, ultrasound reception section 4, signal processing section 5, image generation section 6, system control section 7, operation section 8, input/output section 9, external power source connection detection section 13, timer 17, and display 113.

Ultrasound diagnostic apparatus 101 receives supply of electric power from at least one of first battery 116, second battery 110, and the AC adapter (the first power source section or the second power source section) connected via AC adapter connection section 12. When ultrasound diagnostic system 100 is activated by a user pressing power source button 16, power source control section 14 controls switch section 18 and power source supplying section 15 such that electric power is supplied to each constituent section in ultrasound diagnostic apparatus 101 via power source supplying section 15.

AC adapter connection section 12 is connected to the AC adapter (the first power source section or the second power source section). Here, the AC adapter as the first power source section is an external power source that can supply electric power with a normal output electric current (e.g., seven amperes). Furthermore, the AC adapter as the second power source section is an external power source that can supply electric power with a large electric current (e.g., ten amperes) whose output electric current amount is larger than usual. Here, it is desirable that the maximum electric current supply amount of the AC adapter as the second power source section is at least 1.3 times the maximum electric current supply amount of the AC adapter as the first power source section. The maximum electric current supply amount is an example of information on electric power supplying capability. AC adapter connection section 12 performs AC-DC conversion on electric power (AC electric power) supplied from the AC adapter and supplies the converted electric power (DC electric power) to switch section 18. Note that, it is assumed that both the AC adapter as the first power source section and the AC adapter as the second power source section have a constant power source voltage (e.g., 15 volts). Since the AC adapter as the second power source section has a larger output electric current than the AC adapter as the first power source section, the AC adapter as the second power source section has a larger electric power to be supplied than the AC adapter as the first power source section.

AC adapter connection section 12, switch section 18, and power source control section 14 are connected such that electric power from the external power source, that is, the AC adapter is transmitted in this order. That is, power source control section 14 is connectable to both the AC adapter as the first power source section, whose output electric current is normal, and the AC adapter as the second power source section, whose output electric current is large, via switch section 18 and AC adapter connection section 12.

First battery 116 and second battery 110 are rechargeable power sources for ultrasound diagnostic apparatus 101. First battery 116 and second battery 110 are charged by receiving supply of electric power from the external power source via switch section 18. When the charged electric power of first battery 116 and the charged electric power of second battery 110 are discharged, the charged electric power of first battery 116 and the charged electric power of second battery 110 are supplied to power source control section 14 directly or via switch section 18. The electric power discharged from first battery 116 and the electric power discharged from second battery 110 are transmitted from power source control section 14 to power source supplying section 15 in a case where the electric power discharged from first battery 116 and the electric power discharged from second battery 110 are used to drive ultrasound diagnostic apparatus 101. In a case where the electric power discharged from first battery 116 and the electric power discharged from second battery 110 are used for charging second battery 110 and first battery 116, the electric power discharged from first battery 116 and the electric power discharged from second battery 110 are transmitted from power source control section 14 to second battery 110 and first battery 116 via switch section 18.

First battery 116 includes remaining capacity acquisition section 116a, which acquires information on the remaining capacity of first battery 116, and deterioration degree acquisition section 116b, which acquires information on the deterioration degree of first battery 116. In addition, second battery 110 includes remaining capacity acquisition section 110a, which acquires information on the remaining capacity of second battery 110, and deterioration degree acquisition section 110b, which acquires information on the deterioration degree of second battery 110.

Each of remaining capacity acquisition sections 110a and 116a is constituted by, for example, a voltage sensor that detects the voltage between terminals of the corresponding battery. Each of deterioration degree acquisition sections 110b and 116b is constituted by, for example, an MPU that measures a temporal change in the value of the voltage between terminals of the corresponding battery when the battery is fully charged. Information signals acquired by remaining capacity acquisition sections 110a and 116a and deterioration degree acquisition sections 110b and 116b are transmitted from each battery to power source control section 14. Information signals acquired by remaining capacity acquisition sections 110a and 116a and deterioration degree acquisition sections 110b and 116b are used for switching control at power source control section 14. The switching control is switching control for switching between charging control on first battery 116 and charging control on second battery 110 and switching control for switching between discharging control on first battery 116 and discharging control on second battery 110.

External power source connection detection section 13 monitors whether electric power is supplied from the AC adapter to AC adapter connection section 12, and transmits an identification signal indicating the presence or absence of the power source connection (power source connection presence/absence identification signal) to power source control section 14. External power source connection detection section 13 is constituted by a voltage sensor that detects an output voltage of AC adapter connection section 12, or the like. The identification signal indicating the presence or absence of the power source connection is, specifically, a signal that makes it possible to determine whether or not the apparatus is electrically connected to the AC adapter by outputting different signals depending on whether or not electric power is supplied from the AC adapter.

The power source connection presence/absence identification signal is used for switching control for switching between charging control on first battery 116 and charging control on second battery 110 and switching control for switching between discharging control on first battery 116 and discharging control on second battery 110 at power source control section 14. In a case where electric power is supplied from the AC adapter, for example, in the discharging control, electric power from the AC power source may be transmitted to power source supplying section 15 without discharging electric power from either first battery 116 or second battery 110 (pass-through operation). Further, in this case, one of first battery 116 and second battery 110 may be charged in the charging control. In a case where no electric power is supplied from the AC adapter, for example, in the discharging control, electric power may be discharged from either first battery 116 or second battery 110 and transmitted to power source supplying section 15, and in this case, charging of both first battery 116 and second battery 110 may be stopped in the charging control.

Furthermore, when the AC adapter is connected to AC adapter connection section 12, external power source connection detection section 13 detects information on the electric power supplying capability of the connected AC adapter. External power source connection detection section 13 is an example of a detection section. The detected information is notified from external power source connection detection section 13 to power source control section 14, and is utilized for power source control at power source control section 14. In the present embodiment, external power source connection detection section 13 detects the electric current amount of the connected AC adapter. As a method of detecting the information on the electric power supplying capability of the connected AC adapter, for example, information on the standard of the AC adapter (e.g., the maximum amount of electric current to be supplied) may be superimposed on the direct current supplied by the connected AC adapter, and external power source connection detection section 13 may read this information. Alternatively, a load may be placed on a path through which electric power is received from the AC adapter, and external power source connection detection section 13 may detect the electric current amount based on a voltage drop before and after the load.

Ultrasound probe 1 includes a transducer, converts a transmission electric signal from ultrasound transmission section 2 into ultrasound, and transmits, toward a subject, ultrasound emitted from the transducer in a state in which ultrasound probe 1 is in contact with a surface of the subject. Then, ultrasound probe 1 receives reflected ultrasound from the subject, converts the reflected ultrasound into a reception electric signal by the transducer, and supplies the reception electric signal to ultrasound reception section 4.

Ultrasound transmission section 2 is a signal processing circuit that supplies a transmission electric signal for transmitting ultrasound to the transducer in ultrasound probe 1. Transmission control section 3 is a control circuit that controls a transmission electric signal for transmitting ultrasound from ultrasound probe 1 to a subject.

Ultrasound reception section 4 is a signal processing circuit that generates a reception signal by performing AC-DC conversion, gain adjustment, and/or the like on a reception electrical signal received from ultrasound probe 1.

Signal processing section 5 is a signal processing circuit that performs various kinds of digital signal processing such as log compression and edge enhancement on the reception signal generated by ultrasound reception section 4, and outputs the reception signal subjected to the various kinds of digital processing to image generation section 6.

Image generation section 6 performs various kinds of image conversion on data outputted by signal processing section 5 to generate image data of an ultrasound image, and transmits the image data to display 113 to cause display 113 to display the ultrasound image. Image generation section 6 is constituted by, for example, a hardware processor such as an MPU, and a memory such as a non-volatile memory or a hard disk.

System control section 7 is connected to ultrasound transmission section 2, transmission control section 3, ultrasound reception section 4, signal processing section 5, image generation section 6, operation section 8, input/output section 9, power source control section 14, power source supplying section 15, and display 113, and performs system control on each section or between sections. System control section 7 is constituted by a memory, such as a non-volatile memory or a hard disk, that stores a program for system control and data associated with the program, and a hardware processor such as an MPU that executes the program.

Operation section 8 receives an input operation of a user and outputs an operation signal to system control section 7. Operation section 8 is, for example, a button, a trackball, a rotary encoder, a touch screen, or the like. Operation section 8 is also generally referred to as a console. Input/output section 9 is used for connection to an external device or the like. Input/output section 9 performs input and output of an image signal, such as storing of the image data to be generated by image generation section 6 in the external device and reading of the image data in the external device, under the control of system control section 7.

Display 113 displays the image data generated by image generation section 6 as an image. In addition, display 113 also displays various setting screens under the control of system control section 7. Furthermore, display 113 can also display the respective replacement times of first battery 116 and second battery 110, which are calculated by system control section 7. In a case where operation section 8 includes a touch screen, the touch screen is provided in display 113.

### Charge/Discharge Switching Mechanism of Ultrasound Diagnostic System 100

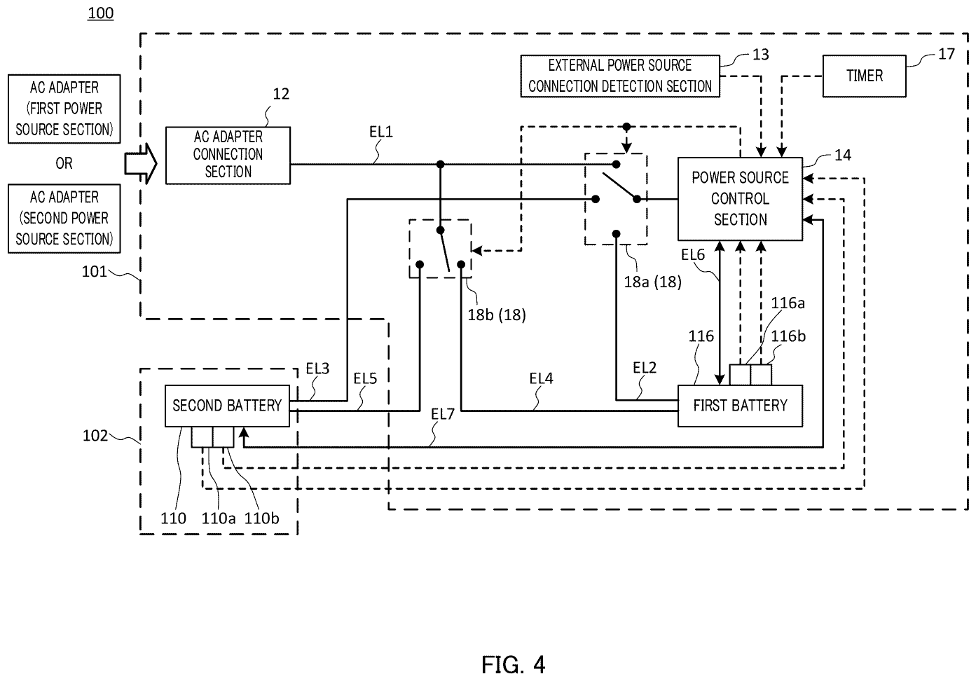

FIG. 4 is a block diagram illustrating a charge/discharge switching mechanism of ultrasound diagnostic system 100 according to the present embodiment. The charge/discharge switching mechanism of ultrasound diagnostic system 100 includes power source control section 14 of ultrasound diagnostic apparatus 101, first battery 116 of ultrasound diagnostic apparatus 101, and second battery 110 of cart 102. The charge/discharge switching mechanism of ultrasound diagnostic system 100 further includes AC adapter connection section 12b of ultrasound diagnostic apparatus 101 and switch section 18 of ultrasound diagnostic apparatus 101. The charge/discharge switching mechanism of ultrasound diagnostic system 100 further includes remaining capacity acquisition section 116a of first battery 116 and deterioration degree acquisition section 116b of first battery 116. The charge/discharge switching mechanism of ultrasound diagnostic system 100 further includes remaining capacity acquisition section 110a of second battery 110, deterioration degree acquisition section 110b of second battery 110, external power source connection detection section 13, and timer 17.

Switch section 18 includes first switching element 18a and second switching element 18b. First switching element 18a and second switching element 18b are constituted by a transistor, a metal oxide semiconductor field effect transistor (MOSFET), or the like. Switching signals to first switching element 18a and second switching element 18b are transmitted from power source control section 14.

First switching element 18a is selectively switched to one of three paths. Three paths are path EL1 connecting AC adapter connection section 12 and power source control section 14, path EL2 connecting first battery 116 and power source control section 14, and path EL3 connecting second battery 110 and power source control section 14. Note that, when first switching element 18a is switched to path EL1 to connect AC adapter connection section 12 and power source control section 14, a pass-through operation is executed in which electric power from the AC adapter is directly supplied to the execution section without passing through first battery 116 and second battery 110.

Second switching element 18b connects a connection path, which connects AC adapter connection section 12 and second switching element 18b, to a middle of path EL1 connecting AC adapter connection section 12 and first switching element 18a. Second switching element 18b is selectively switched to one of path EL4 connecting AC adapter connection section 12 and first battery 116 and path EL5 connecting AC adapter connection section 12 and second battery 110. In a case where first battery 116 or second battery 110 is charged, second switching element 18b is switched such that electric power supplied from the external power source becomes either electric power for charging first battery 116 or electric power for charging second battery 110. Note that, in a case where neither first battery 116 nor second battery 110 is charged, second switching element 18b may be in an off state such that electric power supplied from the external power source does not become electric power for charging first battery 116 nor electric power for charging second battery 110.

Note that, the switching among the various paths at switch section 18 is automatically performed according to the states of first battery 116 and second battery 110, or the like, but may be manually performed as appropriate. A case where the path switching is automatically performed is preferred since the charging control switching and the discharging control switching become efficient.

In addition, ultrasound diagnostic apparatus 101 is separately provided with path EL6 and path EL7. Path EL6 is a path for transmission of discharged electric power from first battery 116 to power source control section 14 or transmission (distribution) of part of electric power supplied from the external power source from power source control section 14 to first battery 116. Path EL7 is a path for transmission of discharged electric power from second battery 110 to power source control section 14 or transmission (distribution) of part of electric power supplied from the external power source from power source control section 14 to second battery 110.

Power source control section 14 performs both switching control (charging switching control) for switching between charging control on first battery 116 and charging control on second battery 110 and switching control (discharging switching control) for switching between discharging control on first battery 116 and discharging control on second battery 110.

Power source control section 14 performs, for example, the following switching control on the basis of the information on the remaining capacity of first battery 116 and the information on the remaining capacity of second battery 110, which are acquired by remaining capacity acquisition sections 110a and 116a.

For example, when the remaining capacity of second battery 110 is smaller than the remaining capacity of first battery 116, power source control section 14 performs the charging control switching and the discharging control switching such that electric power supplied from the external power source and electric power discharged from first battery 116 are utilized to charge second battery 110.

During the switching control for charging second battery 110, first switching element 18a is switched to path EL3 connecting second battery 110 and power source control section 14 under the control of power source control section 14. The electric power discharged from first battery 116 is transmitted via path EL6 and is used as the driving electric power for ultrasound diagnostic apparatus 101, and is sequentially transmitted via paths EL6 and EL3 and is used as electric power for charging second battery 110. Second switching element 18b is switched to path EL5 connecting AC adapter connection section 12 and second battery 110, and the electric power supplied from the external power source is transmitted via EL5 and is used as electric power for charging second battery 110.

In this way, power source control section 14 can perform the switching control by referring to the remaining capacity of at least one of first battery 116 and second battery 110. The switching control is performed such that the electric power discharged from one of first battery 116 and second battery 110 is used for charging the other of first battery 116 and second battery 110 together with the electric power supplied from the external power source. Here, one of the above-described batteries may be a battery having a small remaining capacity or a battery having a large remaining capacity. Whether one of the above-described batteries is used as the battery having a small remaining capacity or the battery having a large remaining capacity may be determined on the basis of the deterioration degrees of first battery 116 and second battery 110 and/or non-use times (durations of a non-use state) of first battery 116 and second battery 110. In addition, whether the remaining capacity is large or small may be determined by comparing both the remaining capacities of first battery 116 and second battery 110 with each other, or may be determined by comparing the remaining capacity of at least one of first battery 116 and second battery 110 with a specified value set in advance.

Power source control section 14 performs, for example, the following switching control based on information on the deterioration degree of first battery 116 and information on the deterioration degree of second battery 110, which are acquired by deterioration degree acquisition sections 110b and 116b.

For example, when the deterioration degree of first battery 116 is higher than the deterioration degree of second battery 110, power source control section 14 performs the charging control switching and the discharging control switching such that electric power supplied from the external power source and electric power discharged from first battery 116 are utilized to charge second battery 110.

During the switching control for charging second battery 110, first switching element 18a is switched to path EL3 connecting second battery 110 and power source control section 14 under the control of power source control section 14. The electric power discharged from first battery 116 is transmitted via path EL6 and is used as the driving electric power for ultrasound diagnostic apparatus 101, and is sequentially transmitted via paths EL6 and EL3 and is used as electric power for charging second battery 110. Second switching element 18b is switched to path EL5 connecting AC adapter connection section 12 and second battery 110, and the electric power supplied from the external power source is transmitted via EL5 and is used as electric power for charging second battery 110.

In this way, power source control section 14 can perform the switching control by referring to the deterioration degree of at least one of first battery 116 and second battery 110. The switching control is performed such that the electric power discharged from one of first battery 116 and second battery 110 is used for charging the other of first battery 116 and second battery 110 together with the electric power supplied from the external power source. Here, one of the above-described batteries may be a battery having a low deterioration degree or a battery having a high deterioration degree. Whether one of the above-described batteries is used as the battery having a low deterioration degree or the battery having a high deterioration degree may be determined on the basis of the remaining capacities of first battery 116 and second battery 110 and/or non-use times of first battery 116 and second battery 110. In addition, whether the deterioration degree is low or high may be determined by comparing both the deterioration degrees of first battery 116 and second battery 110 with each other, or may be determined by comparing the deterioration degree of at least one of first battery 116 and second battery 110 with a specified value set in advance.

Power source control section 14 performs, for example, the following switching control on the basis of information indicated in the power source connection presence/absence identification signal from external power source connection detection section 13.

For example, when electric power is supplied from the external AC power source, power source control section 14 performs the charging control switching and the discharging control switching. The charging control switching and the discharging control switching are performed based on the remaining capacities, the deterioration degrees, and non-use times of first battery 116 and second battery 110. The charging control switching and the discharging control switching are performed such that electric power supplied from the external power source and electric power discharged from one of first battery 116 and second battery 110 are utilized to charge the other of first battery 116 and second battery 110. In addition, in a case where no electric power is supplied from the external power source, power source control section 14 performs the charging control switching and the discharging control switching such that none of first battery 116 and second battery 110 is charged and electric power discharged from one of first battery 116 and second battery 110 is utilized as it is as the driving power source for ultrasound diagnostic system 100.

### Power Source Control according to Electric Power Supplying Capability of AC Adapter

Hereinafter, power source control according to the electric power supplying capability of the AC adapter will be described. Here, as an example of the power source control, electric power distribution control to the execution section and first battery 116 for, while driving the execution section by the pass-through operation, charging first battery 116 in parallel will be described as an example. The execution section is, as described above, a portion that includes ultrasound transmission section 2, ultrasound reception section 4, and the like, and executes ultrasound diagnosis.

As a premise of the following description, it is assumed that the electric current for (electric current discharged from) first battery 116 is five amperes and the driving electric current of the execution section is five amperes at the time of full operation. Further, it is assumed that the electric current amount of electric power supplied from the AC adapter as the first power source section is seven amperes, and the electric current amount of electric power supplied from the AC adapter as the second power source section is ten amperes.

Note that, as a variation, the charging target may be second battery 110 instead of first battery 116, or may be both first battery 116 and second battery 110.

In the following description, for simplicity, it is assumed that discharging from second battery 110 to first battery 116 is not performed for charging first battery 116.

When the AC adapter as the first power source section is connected to AC adapter connection section 12, external power source connection detection section 13 detects the electric current amount of electric power supplied from the connected AC adapter. Here, since the connected AC adapter is the first power source section, the electric current amount is seven amperes. That is, external power source connection detection section 13 detects that the electric current amount is seven amperes.

When power source control section 14 is notified that the electric current amount of the AC adapter is seven amperes, power source control section 14 determines the ratio of electric power distribution to the execution section and the ratio of electric power distribution to first battery 116 according to the notified ampere value. In this example, power source control section 14 determines that the ratio of electric power distribution to the execution section is 5/7 and the ratio of electric power distribution to first battery 116 is 2/7. Then, power source control section 14 transmits five amperes out of seven amperes to power source supplying section 15 (distributes five amperes out of seven amperes to the execution section) for driving the execution section based on the ratio of electric power distribution to the execution section. In addition, power source control section 14 transmits the remaining two amperes to first battery 116 (distributes the remaining two amperes to the first battery) for charging first battery 116 based on the ratio of electric power distribution to the execution section.

In this way, power source control section 14 supplies an electric current supply amount exceeding half of the electric current amount of the electric power supplied from the AC adapter to the execution section to distribute the electric power to the execution of ultrasound diagnosis with priority over the charging of first battery 116. Thus, the execution section can be stably operated while first battery 116 is charged. On the other hand, as for the charging of first battery 116, the electric current for charging is reduced as compared with a case where the execution section is in the non-operation state. In a case where the execution section is in the non-operation state, the charging of first battery 116 can be executed with five amperes at the fastest charging, whereas in a case where the execution section is in the operation state, the charging of first battery 116 cannot be executed at the fastest charging. Here, since the electric current for charging is two amperes, it is ⅖ with respect to five amperes at the fastest charging. In other words, the charging time of first battery 116 is 2.5 times longer by simple calculation.

Here, when a user desires charging in a shorter time, the user may connect the AC adapter as the second power source section capable of supplying electric power with a larger electric current to AC adapter connection section 12 in place of the AC adapter as the first power source section. The AC adapter as the second power source section has an electric current amount of ten amperes as described above.

The purpose of using the AC adapter as the second power source section is to enable charging in a shorter time with respect to the AC adapter as the first power source section. In a case where the electric current amounts of both the adapters are not significantly different, the AC adapter as the second power source section cannot fulfill this purpose. Therefore, as described above, the maximum electric current supply amount of the AC adapter as the second power source section is desirably at least 1.3 times the maximum electric current supply amount of the AC adapter as the first power source section.

When the AC adapter as the second power source section is connected to AC adapter connection section 12, external power source connection detection section 13 detects that the electric current amount of electric power supplied from the connected AC adapter is ten amperes.

When power source control section 14 is notified that the electric current amount of the AC adapter is ten amperes, power source control section 14 determines the ratio of electric power distribution to the execution section and the ratio of electric power distribution to first battery 116 according to the notified amperage. In this example, power source control section 14 determines that both the ratio of electric power distribution to the execution section and the ratio of electric power distribution to first battery 116 are 5/10 (=½). Then, power source control section 14 transmits five amperes out of ten amperes each time to power source supplying section 15 (distributes five amperes out of ten amperes each time to the execution section) for driving the execution section based on the determined ratio of electric power distribution. In addition, power source control section 14 transmits electric power to first battery 116 (distributes electric power to the first battery) for charging first battery 116 based on the determined ratio of electric power distribution. As a result, it is possible to supply the execution section with electric power required during its full operation and to supply first battery 116 with electric power required for the fastest charging.

Assuming that an attempt is made to increase the ratio of electric power distribution to first battery 116 with the electric current amount of the AC adapter remaining at seven amperes, it is necessary to reduce the ratio of electric power distribution to the execution section by that much. In this case, it is difficult to maintain the full operation state of the execution section, and the execution section needs to be shifted to a state in which power consumption is suppressed, such as a power save mode. In the present embodiment, however, by increasing the ratio of electric power distribution to first battery 116 in a case where the electric current amount of the AC adapter has increased, it becomes unnecessary to reduce the ratio of electric power distribution to the execution section. As a result, it is possible to achieve both the full operation state of the execution section and the fastest charging of first battery 116.

As described above, according to the present embodiment, ultrasound diagnostic apparatus 101 includes the execution section, first battery 116, and power source control section 14 that are incorporated in apparatus main body 115. Power source control section 14 is connectable to both the first power source section including an AC adapter and the second power source section including an AC adapter and having suppliable electric power (that is, an electric current supply amount (provided that the source voltage is constant)) larger than that of the first power source section. Power source control section 14 performs control such that the amount(s) of electric power to be supplied (electric current value) to the execution section and/or first battery 116 is/are varied according to the electric power supplying capability of the connected power source section.

That is, in the present embodiment, it is configured such that AC adapters having different suppliable electric powers can be connected to power source control section 14. Then, it is configured such that power source control section 14 causes the amount(s) of electric power to be supplied to the execution section and/or first battery 116 to be varied according to the electric power supplying capability of the connected power source section. Therefore, a user can select a charging time that suits the way of use of the user, by appropriately selecting the AC adapter to be connected. That is, flexible battery charging according to a use situation of a user has become possible.

Furthermore, in the present embodiment, external power source connection detection section 13 detects information on the electric power supplying capability of the connected power source section, specifically, the electric current amount of the connected power source section. Then, power source control section 14 performs control based on the detected information such that the amount(s) of electric power to be supplied to the execution section and/or first battery 116 is/are varied. Since it is ensured that the electric power supplying capability of the power source section is detected, a change in electric power to be supplied due to the replacement of the power source section to be connected can be used as a trigger for control such that the amount(s) of electric power to be supplied to the execution section and/or first battery 116 is/are varied.

### Embodiment 2

Hereinafter, Embodiment 2 of the present invention will be described. Note that. the present embodiment is basically the same as Embodiment 1. Therefore, in the present embodiment, constituent elements common to those in Embodiment 1 are denoted by the same reference signs as those in Embodiment 1 denote, and detailed descriptions thereof will be omitted. The present embodiment is different from Embodiment 1 in that there are two AC adapter connection sections and a maximum of two AC adapters can be connected. Hereinafter, differences from Embodiment 1 will be mainly described.

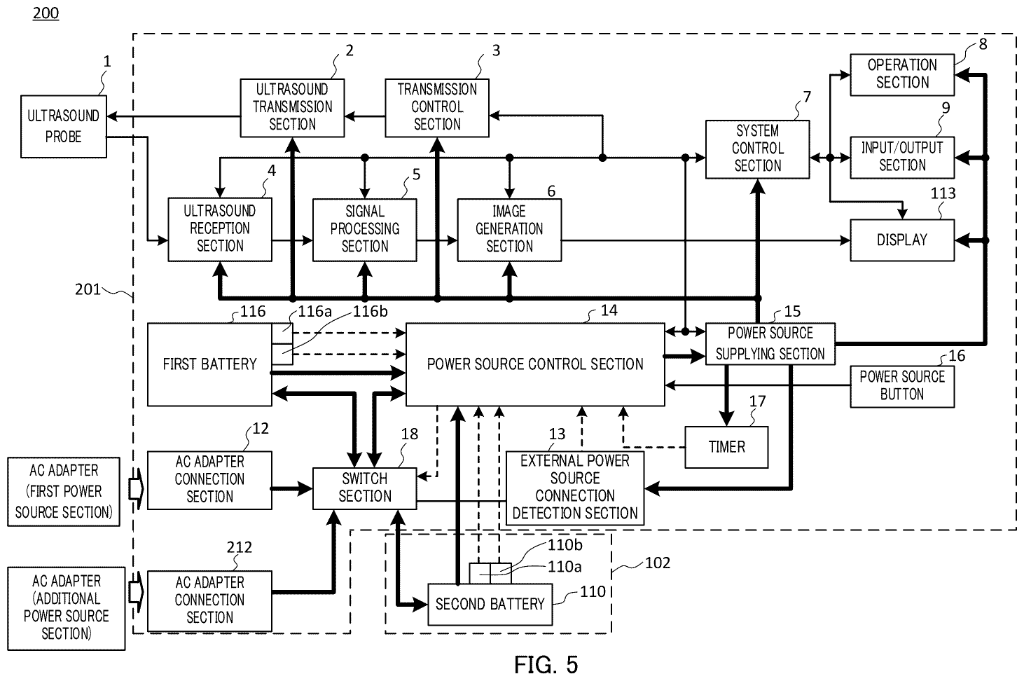

FIG. 5 is a block diagram illustrating an internal configuration of an ultrasound diagnostic system including an ultrasound diagnostic apparatus according to Embodiment 2. As illustrated, ultrasound diagnostic apparatus 201 according to the present embodiment is obtained by adding AC adapter connection section 212 to the configuration of ultrasound diagnostic apparatus 101 described in Embodiment 1. Ultrasound diagnostic system 200 includes ultrasound diagnostic apparatus 201 and cart 102.

Power source control section 14 is supplied with electric power from one or more AC adapters via switch section 18. In the first case, an AC adapter is connected to AC adapter connection section 12 between AC adapter connection portions 12 and 212, and electric power is supplied from one AC adapter thereto. The AC adapter to be connected to AC adapter connection section 12 is an external power source that can supply electric power with a normal output electric current (e.g., seven amperes) and is an example of the first power source section. In the second case, AC adapters are connected to both AC adapter connection sections 12 and 212, and electric power is supplied from the two AC adapters thereto. The AC adapter to be connected to AC adapter connection section 212 is an external power source that can supply electric power with a normal output electric current (e.g., seven amperes) and is an example of an additional power source section. When these two AC adapters are connected at the same time, the total suppliable electric power is twice as large as in a case where only one AC adapter is connected. That is, in the present embodiment, a plurality of AC adapters in which the AC adapter as the first power source section and the AC adapter as the additional power source section are combined is an example of the second power source section.

External power source connection detection section 13 detects the number of AC adapters connected to AC adapter connection sections 12 and 212 as information on the electric power supplying capability of the AC adapters. As a specific detection method, a load may be disposed in each of two paths through which electric power is received from the two AC adapters, respectively, and external power source connection detection section 13 may detect the number of connected AC adapters based on the number of loads in which a voltage drop has occurred.

When only the AC adapter as the first power source section is connected to AC adapter connection section 12, external power source connection detection section 13 detects that the number of the connected AC adapters is one.

When power source control section 14 is notified that the number of connected AC adapters is one, power source control section 14 determines based on the notified number that the total amount of electric current to be supplied from the external power source is seven amperes. Then power source control section 14 determines the ratio of electric power distribution to the execution section and the ratio of electric power distribution to first battery 116 according to the determined ampere value. In this example, power source control section 14 determines that the ratio of electric power distribution to the execution section is 5/7 and the ratio of electric power distribution to first battery 116 is 2/7. Then, power source control section 14 transmits five amperes out of seven amperes to power source supplying section 15 (distributes five amperes out of seven amperes to the execution section) for driving the execution section based on the ratio of electric power distribution to the execution section. In addition, power source control section 14 transmits the remaining two amperes to first battery 116 (distributes the remaining two amperes to the first battery) for charging first battery 116 based on the ratio of electric power distribution to the execution section. When the electric current for charging becomes two amperes, which is ⅖ with respect to five amperes with which the fastest charging is possible, the charging time of first battery 116 becomes 2.5 times longer by simple calculation.

Here, when a user desires charging in a shorter time, the user may connect the AC adapter as the additional power source section to AC adapter connection section 212 in addition to the AC adapter as the first power source section. In this case, the amount of electric current to be supplied from the two AC adapters as the second power source section obtained by combining the first power source section and the additional power source section is 14 amperes.

When the two AC adapters as the second power source section are connected to AC adapter connection sections 12 and 212, external power source connection detection section 13 detects that the number of connected AC adapters is two.

When power source control section 14 is notified that the number of connected AC adapters is two, power source control section 14 determines based on the notified number that the total amount of electric current to be supplied from the external power sources is 14 amperes. Then, power source control section 14 determines the ratio of electric power distribution to the execution section and the ratio of electric power distribution to first battery 116 according to the determined amperage. In this example, power source control section 14 determines that both the ratio of electric power distribution to the execution section and the ratio of electric power distribution to first battery 116 are 5/14. Next, based on the determined ratio of electric power distribution, power source control section 14 transmits five amperes of 14 amperes each time power source supplying section 15 (distributes five amperes of 14 amperes each time to the execution sections) for driving the execution section based on the determined ratio of electric power distribution. In addition, power source control section 14 transmits electric power to first battery 116 (distributes electric power to the first battery) for charging first battery 116 based on the determined ratio of electric power distribution. As a result, it is possible to supply the execution section with electric power required during its full operation and to supply first battery 116 with electric power required for the fastest charging.

As in the present embodiment, even in a case where it is configured such that the number of connected AC adapters is detected as the information on the electric power supplying capability/capabilities of the external power source(s), a user can select a charging time that suits the way of use of the user. by increasing the number of AC adapters to be connected. That is, flexible battery charging according to a use situation of a user has become possible.

Furthermore, in the present embodiment, external power source connection detection section 13 detects the information on the electric power supplying capability/capabilities of the connected power source section(s), specifically, the number of power source sections to be connected. Then, power source control section 14 performs control based on the detected information such that the amount(s) of electric power to be supplied to the execution section and/or first battery 116 is/are varied. Since it is ensured that the electric power supplying capability/capabilities of the power source section(s) is/are detected, a change(s) in the electric power supplying capability/capabilities due to an increase in the number of connected power source sections can be used as a trigger for control such that the amount(s) of electric power to be supplied to the execution section and/or first battery 116 is/are varied.

Note that, in the present embodiment, the surplus four amperes may be utilized for charging second battery 110 incorporated in cart 102. In this case, the ratio of electric power distribution to second battery 110 increases from 0/14 to 4/14. That is, power source control section 14 can perform control such that the amount(s) of electric power to be supplied to the execution section. first battery 116, and/or second battery 110 is/are varied according to the electric power supplying capability/capabilities of the connected power source section(s).

### Embodiment 3

Embodiment 3 of the present invention will be described. The present embodiment is basically the same as Embodiments 1 and 2. Therefore, in the present embodiment, constituent elements common to those in Embodiments 1 and 2 are denoted by the same reference sign as those in Embodiments 1 and 2. and detailed descriptions thereof will be omitted. The present embodiment is particularly similar to Embodiment 2. Specifically, the present embodiment is different from Embodiment 2 only in that the second AC adapter connection section included in ultrasound diagnostic apparatus 201 in Embodiment 2 is included in cart 302. That is, the configuration of ultrasound diagnostic apparatus 301 according to the present embodiment is obtained by removing AC adapter connection section 212 from the configuration of ultrasound diagnostic apparatus 201 according to Embodiment 2. Ultrasound diagnostic system 300 is a combination of ultrasound diagnostic apparatus 301 and cart 302. The operation and effect of the present embodiment are the same as those of Embodiment 2, and thus, detailed descriptions thereof will be omitted here.

### Embodiment 4

Embodiment 4 of the present invention will be described. The present embodiment is basically the same as Embodiments 1 to 3. Therefore, in the present embodiment, constituent elements common to those in Embodiments 1 to 3 are denoted by the same reference signs as those in Embodiments 1 to 3, and detailed descriptions thereof will be omitted. The present embodiment is relatively similar to Embodiment 1. Specifically, in the present embodiment, ultrasound diagnostic apparatus 401 incorporates an AC adapter itself as AC adapter section 412, instead of AC adapter connection section 12 to which an AC adapter is connected. Accordingly in the present embodiment, power source connection detection section 413 is provided instead of external power source connection detection section 13. Ultrasound diagnostic system 400 is a combination of ultrasound diagnostic apparatus 401 and cart 102.

AC adapter section 412 supplies electric power when a power source cable is connected thereto and a power source plug of the power source cable is connected to a commercial power source. AC adapter section 412 is capable of switching suppliable electric powers (the suppliable amounts of electric current), for example, by a user operation at operation section 8.

Power source connection detection section 413 monitors whether or not electric power is being supplied from AC adapter section 412, thereby transmitting an identification signal indicating the presence or absence of power source connection (a power source connection presence/absence identification signal) to power source control section 14. Power source connection detection section 413 is constituted by a voltage sensor that detects an output voltage of AC adapter section 412, or the like. The identification signal indicating the presence or absence of the power source connection is, specifically, a signal that makes it possible to determine whether or not electrical connection to an external power source occurs, by outputting different signals depending on whether or not electric power is supplied from AC adapter section 412.

The power source connection presence/absence identification signal is used for switching control for switching between charging control on first battery 116 and charging control on second battery 110 and switching control for switching between discharging control on first battery 116 and discharging control on second battery 110 at power source control section 14. In a case where electric power is supplied from AC adapter section 412, for example, in the discharging control, electric power from the AC power source may be transmitted to power source supplying section 15 without discharging electric power from either first battery 116 or second battery 110 (pass-through operation), and in the discharging control, either first battery 116 or second battery 110 may be charged. In addition, in a case where no electric power is supplied from AC adapter section 412, for example, in the discharging control, electric power may be discharged from either first battery 116 or second battery 110 and transmitted to power source supplying section 15, and charging of both first battery 116 and second battery 110 may be stopped in the charging control.

Furthermore, when AC adapter section 412 is connected to the commercial power source via the power source cable, power source connection detection section 413 detects information on the electric power supplying capability of AC adapter section 412 connected to the commercial power source. Power source connection detection section 413 is an example of the detection section. The detected information is notified from power source connection detection section 413 to power source control section 14, and is utilized for power source control at power source control section 14. In the present embodiment, power source connection detection section 413 detects the electric current amount of AC adapter section 412. As a method of detecting the information on the electric power supplying capability of AC adapter section 412, for example, a load may be placed on a path through which power is received from AC adapter section 412, and power source connection detection section 413 may detect the electric current amount based on a voltage drop before and after the load.

Here, it is assumed that the electric current amount detected by power source connection detection section 13 when AC adapter section 412 is connected to the commercial power source is seven amperes. In other words, at this time, AC adapter section 412 functions as the first power source section to supply electric power with the normal electric current amount.

When power source control section 14 is notified that the electric current amount of AC adapter section 412 is seven amperes, power source control section 14 determines the ratio of electric power distribution to the execution section and the ratio of electric power distribution to first battery 116 according to the notified ampere value. Here, as an example, power source control section 14 determines that the ratio of electric power distribution to the execution section is 5/7 and the ratio of electric power distribution to first battery 116 is 2/7. Then, power source control section 14 transmits five amperes out of seven amperes to power source supplying section 15 (distributes five amperes out of seven amperes to the execution section) for driving the execution section based on the ratio of electric power distribution to the execution section. In addition, power source control section 14 transmits the remaining two amperes to first battery 116 (distributes the remaining two amperes to the first battery) for charging first battery 116 based on the ratio of electric power distribution to the execution section.

In this way, power source control section 14 supplies an electric current supply amount exceeding half of the electric current amount of the electric power supplied from AC adapter section 412 to the execution section to distribute the electric power to the execution of ultrasound diagnosis with priority over the charging of first battery 116. Thus, the execution section can be stably operated while first battery 116 is charged. On the other hand, as for the charging of first battery 116, the electric current for charging is reduced as compared with a case where the execution section is in the non-operation state. In a case where the execution section is in the non-operation state, the charging of first battery 116 can be executed with five amperes at the fastest charging, whereas in a case where the execution section is in the operation state, the charging of first battery 116 cannot be executed at the fastest charging. Here, since the electric current for charging is two amperes, it is ⅖ with respect to five amperes at the fastest charging. In other words, the charging time of first battery 116 is 2.5 times longer by simple calculation.

Here, when a user desires charging in a shorter time, the user may switch AC adapter section 412 such that AC adapter section 412 functions as the second power source section capable of supplying electric power with a larger electric current, for example, ten amperes.

As a result of this switching, power source connection detection section 413 detects that the electric current amount of electric power supplied from AC adapter section 412 is ten amperes.

When power source control section 14 is notified that the electric current amount of AC adapter section 412 is ten amperes, power source control section 14 determines the ratio of power distribution to the execution sections and the ratio of power distribution to first battery 116 according to the notified amperage. In this example, power source control section 14 determines that both the ratio of electric power distribution to the execution section and the ratio of electric power distribution to first battery 116 are 5/10 (=½). The, power source control section 14 transmits five amperes out of ten amperes each time to power source supplying section 15 (distributes five amperes out of ten amperes each time to the execution section) for driving the execution section on the basis of the determined ratio of electric power distribution. In addition, power source control section 14 transmits electric power to first battery 116 (distributes electric power to the first battery) for charging first battery 116 based on the determined ratio of electric power distribution. As a result, it is possible to supply the execution section with electric power required during its full operation and to supply first battery 116 with electric power required for the fastest charging.

Assuming that an attempt is made to increase the ratio of electric power distribution to first battery 116 with the electric current amount of AC adapter section 412 remaining at seven amperes, it is necessary to reduce the ratio of electric power distribution to the execution section by that much. In this case, it is difficult to maintain the full operation state of the execution section, and the execution section needs to be shifted to a state in which power consumption is suppressed, such as a power save mode. In the present embodiment, however, the ratio of power distribution to first battery 116 is increased in a case where the electric current amount of AC adapter section 412 has increased. Thus, it becomes unnecessary to reduce the ratio of electric power distribution to the execution sections. As a result, it is possible to achieve both the full operation state of the execution section and the fastest charging of first battery 116.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus, comprising:
an executor that executes ultrasound diagnosis;
a battery that supplies electric power to the executor; and
a hardware processor that is connectable to both a first power source and a second power source, and performs control such that amounts or an amount of the electric power to be supplied to the executor and/or the battery are/is varied according to electric power supplying capability of a power source that is the first power source or the second power source and has been connected, the first power source supplying the electric power to the executor and the battery, the second power source supplying the electric power to the executor and the battery and having suppliable electric power larger than suppliable electric power of the first power source.

2. The ultrasound diagnostic apparatus according to claim 1, further comprising a sensor that detects information on the electric power supplying capability of the power source that has been connected,
wherein
the hardware processor controls, based on the information that has been detected, the amount or the amount of the electric power to be supplied.

3. The ultrasound diagnostic apparatus according to claim 2, wherein
the information is an electric current amount that is suppliable by the power source to be connected.

4. The ultrasound diagnostic apparatus according to claim 2, wherein
the information is a number of a plurality of the power sources to be connected.

5. The ultrasound diagnostic apparatus according to claim 2, wherein
the hardware processor controls, based on the information, distribution of the electric power to the executor and the battery from the power source that has been connected.

6. The ultrasound diagnostic apparatus according to claim 5, wherein
the hardware processor varies a ratio of electric power distribution to the executor and a ratio of electric power distribution to the battery according to the electric power supplying capability of the power source that has been connected.

7. The ultrasound diagnostic apparatus according to claim 6, wherein
in a case where the first power source is connected, the hardware processor causes the ratio of the electric power distribution to the executor to be larger than the ratio of the electric power distribution to the battery.

8. The ultrasound diagnostic apparatus according to claim 1, wherein
the second power source is constituted by combining a plurality of external power sources.

9. The ultrasound diagnostic apparatus according to claim 1, wherein
the second power source has a maximum electric current supply amount at least 1.3 times a maximum electric current supply amount of the first power source.

10. An ultrasound diagnostic system, comprising:
the ultrasound diagnostic apparatus according to claim 1; and
a cart attachable to and detachable from a main body of the ultrasound diagnostic apparatus, wherein
the main body includes the battery as a first battery,
the cart includes a second battery, and
the hardware processor performs control such that amounts or an amount of the electric power to be supplied to the executor, the first battery, and/or the second battery are/is varied according to the electric power supplying capability of the power source that is the first power source or the second power source and has been connected.

11. A power source control method for an ultrasound diagnostic apparatus, the ultrasound diagnostic apparatus including: an executor; a battery; and a main body, the executor executing ultrasound diagnosis, the battery supplying electric power to the executor, the main body being connectable to both a first power source and a second power source, the first power source supplying the electric power to the executor and the battery, the second power source supplying the electric power to the executor and the battery and having suppliable electric power larger than suppliable electric power of the first power source, the power source control method comprising
performing control such that amounts or an amount of the electric power to be supplied to the executor and/or the battery are/is varied according to electric power supplying capability of a power source that is the first power source or the second power source and has been connected.

12. A non-transitory computer-readable storage medium storing a program for causing the ultrasound diagnostic apparatus to execute the power source control method according to claim 11.
